(19) United States
(12) Patent Application Publication
(10) Pub. No.: US 2016/0015547 A1
(43) Pub. Date: Jan. 21, 2016

(54) ADJUSTABLE BACK SUPPORT DEVICE

(71) Applicant: Victor Toso, St. Paul, MN (US)

(72) Inventor: Victor Toso, St. Paul, MN (US)

(21) Appl. No.: 14/802,647

(22) Filed: Jul. 17, 2015

Related U.S. Application Data
(63) Continuation of application No. 12/202,863, filed on Sep. 2, 2008, now abandoned.
(60) Provisional application No. 60/968,954, filed on Aug. 30, 2007.

(51) Int. Cl. A61F 5/02

(52) U.S. Cl. CPC A61F 5/028

(57) ABSTRACT

Back support devices for use in supporting a lower back region of an individual in a seated position are disclosed. The back support device can include a flexible, double-ended back support member adapted to provide lumbar support to the user's lower back, and a number of elastic knee straps extending from the ends of the back support member each forming a loop that can be attached to the user's knee when in a seated position. The back support device can be equipped with adjustment mechanisms for adjusting the length of the elastic knee straps and/or the length of the back support member. The back support member may further define an interior pouch that can be configured to receive a thermal pack, vibration element and/or other therapeutic device, and which can be used to store the back support device when not in use.

### RELATED APPLICATIONS

This application is a continuation of U.S. application Ser. No. 12/202,863, filed Sep. 2, 2008, which claims priority to U.S. Provisional Application Ser. No. 60/968,954 filed Aug. 30, 2007.

### FIELD

The present invention relates generally to back support devices. More specifically, the present invention pertains to adjustable back support devices for supporting a lower back region of an individual in a seated position.

### BACKGROUND

Many activities require participants to be seated in an upright position for an extended length of time without rigid back support. Examples of recreational activities which require an individual to be seated upright for extended periods of time include canoeing, fishing, hunting, boating, horseback riding, and motorcycling. Other activities such as working at a computer may also require an individual to be seated in an upright position for extended periods of time. Many people find sitting with little or no back support to be very uncomfortable, in some cases distracting from their participation in these activities. Such discomfort may also cause the individual to reduce the amount of time they spend engaged in those activities. For example, on a long motorcycle journey, frequent stops may be required because of discomfort to the lower back. The frequency and severity of discomfort may be increased or exacerbated due to improper posture and/or a weak back structure.

A number of different back support devices have been developed which provide support to a user's lower back. The ability to easily adjust these devices to provide a desired fit or to accommodate for variations in body size is often limited, however, preventing the user from comfortably wearing the device for extended periods of time. In some designs, for example, the back support device may not permit the user to adjust the size of the device in order to give the user a more relaxed fit and/or to improve body posture. In such case, the inability to adjust the device may limit its use. A need therefore exists for back support devices that can be easily adjusted to accommodate for the user's preferences as well as specific body size.

### BRIEF SUMMARY

The present invention relates to devices for supporting a lower back region of an individual in a seated position. A back support device in accordance with an illustrative embodiment can include a flexible, double-ended back support member adapted to provide lumbar support to the user's lower back, and a number of elastic knee straps extending from the ends of the back support member that form several loops that can be attached to the user's knees when the user is in a seated position. The back support member can be constructed from a flexible material that supports the user's lower back when worn. In some embodiments, for example, the flexible material can include elastic webbing formed integrally with or as a separate component from the material forming the elastic knee straps. The elastic webbing can have a strength sufficient to provide back support to the user without the use of additional padding or cushions, although such additional support can be provided, if desired. In some embodiments, the back support member can be made from multiple layers of elastic webbing to increase the amount of back support provided to the user's back.

An adjustment mechanism can be used to adjust the length of the elastic knee straps, allowing the user to vary the size of the loops to provide a more relaxed fit and/or to improve body posture. In one such embodiment, for example, the adjustment mechanism can include a fabric hook and loop type fastener coupled to the ends of the back support member and to the ends of the elastic knee straps. A fastener mechanism can also be provided in some embodiments to permit the user to secure the elastic knee straps together at a location between the first and second ends thereof. When used, the fastener mechanism helps to support the user's knees together while also preventing the loops from inadvertently detaching during use.

A back support adjustment mechanism can be provided in some embodiments to permit the user to adjust the length of the back support member. The back support adjustment mechanism can include, for example, a fabric hook-type fastener attached to a portion of the back support member, which can be configured to mate with several fabric receptor flaps coupled to the ends of the back support member. In use, the length of the back support member can be adjusted by releasing the fabric receptor flaps and then repositioning the flaps relative to the fabric hook to either decrease or increase the length of the back support member, as desired.

The back support member may further define an interior pouch that permits the back support device to be folded upon itself and collapsed for storage when not in use. A number of carrying handles can be provided within the interior pouch to facilitate carrying the back support device once stored within the interior pouch. In some embodiments, the interior pouch may be further configured to receive a therapeutic device such as a thermal pack or vibration element. The interior pouch can be formed from an unsewn interior portion of the back support member, creating an opening through which the therapeutic device can be inserted into the back support member.

Another back support device for supporting a user's lower back may include a back support member. A first knee strap may extend from the back support member. A second knee strap may extend from the back support member and be positioned opposite the first knee strap. A first foot strap may extend from the first knee strap. The first foot strap may include a first foot loop. A second foot strap may extend from the second knee strap. The second foot strap may include a second foot loop.

### BRIEF DESCRIPTION OF THE DRAWINGS

FIG. 1 is a perspective view of a back support device in accordance with an illustrative embodiment having an adjustment mechanism for adjusting several elastic knee straps;

FIG. 2 is another perspective view showing the illustrative back support member of FIG. 1;

FIG. 3 is an enlarged perspective view showing the adjustment mechanism for one of the elastic knee straps of FIG. 2;

FIG. 4 is a perspective view showing the illustrative back support device of FIG. 1 attached to a user;

FIG. 5 is a perspective view of a back support device in accordance with another illustrative embodiment having a back support adjustment mechanism;

FIG. 6 is an enlarged perspective view showing the back support adjustment mechanism of FIG. 5 in greater detail;

FIG. 7 is another enlarged perspective view of the back support adjustment mechanism of FIG. 5 showing the flap detached from the fabric hook;

FIG. 8 is a perspective view showing the rear of the back support device of FIG. 5 attached to a user's body;

FIG. 9 is a perspective view of a back support device in accordance with another illustrative embodiment having an interior pouch;

FIG. 10 is a perspective view showing an interior pouch of the back support device of FIG. 9;

FIG. 11 is a perspective view showing the opening of the interior pouch in a fully expanded position;

FIG. 12 is a perspective view showing the back support device of FIG. 9 folded upon itself within the interior pouch;

FIG. 13 is a perspective view of a back support device in accordance with another illustrative embodiment;

FIG. 14 is a perspective view showing the illustrative back support device of FIG. 13 attached to a user;

FIG. 15 is a rear view of a back support device in accordance with another illustrative embodiment; and

FIG. 16 is a rear view showing the illustrative back support device of FIG. 15 with an accessory panel attached thereto.

### DETAILED DESCRIPTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized.

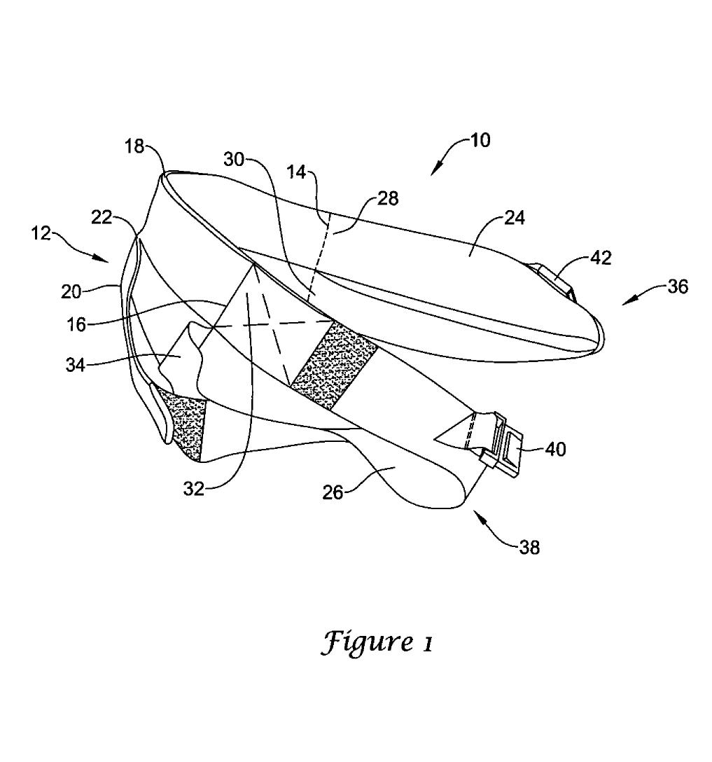

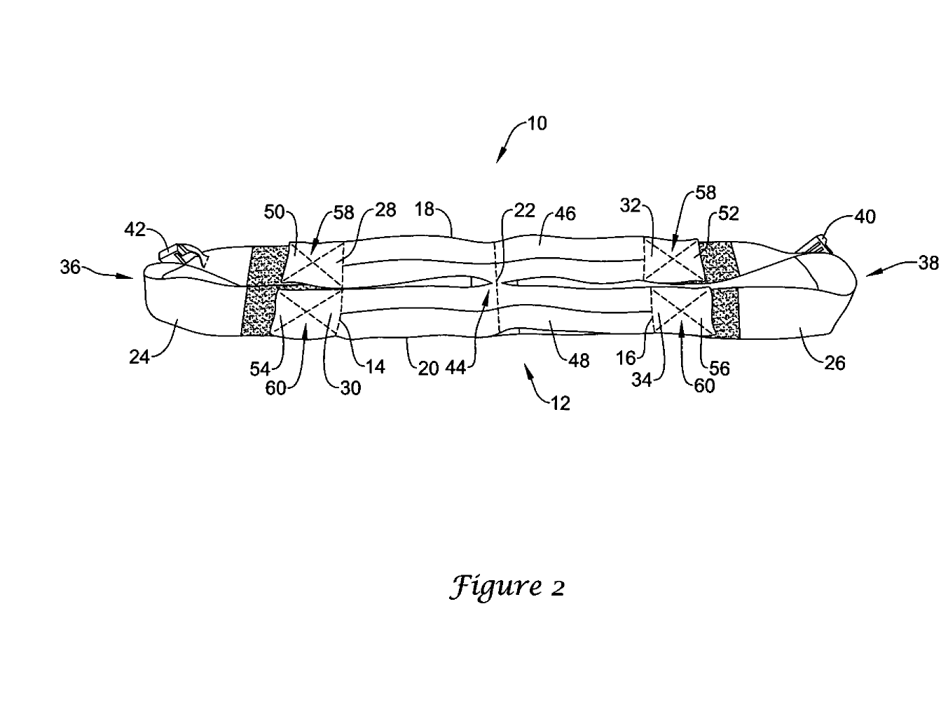

Referring now to FIGS. 1 and 2, a back support device 10 in accordance with an illustrative embodiment having an adjustment mechanism for adjusting several elastic knee straps will now be described. In the illustrative embodiment depicted, the back support device 10 includes a back support member 12 having an elongated and generally rectangular shape adapted to fit about and provide lombar support for the lower portion of a user's back. The back support member 12 can have a double-ended structure including a first end 14, a second end 16, an upper side 18, and a lower side 20. The width of the back support member 12 may be defined generally by the length between the upper 18 and the lower side 20, and can be configured to span substantially the length of the lumbar region of the user's back. The length of the back support member 12, in turn, may be defined generally by the length between the first end 14 and second end 16, and can be configured to span the circumference of the user's lower back.

The length and/or width dimensions of the back support member 12 may vary depending on the size of the user, the amount of back support desired, as well as other factors. An example length of the back support member 12 can be from about 10 inches to about 16 inches, and more specifically about 12 inches to about 14 inches. An example width of the back support member 12 can be from about 2 inches to 8 inches, and more specifically about 4 inches to 6 inches. Other length and/or width dimensions are possible, however. In addition, while the illustrative back support member 12 depicted in FIG. 1 has a substantially rectangular shape, it should be understood that the shape of the member 12 may vary from that shown. In some embodiments, for example, the width of the back support member 12 may be greater towards the center portion 22 of the member 12 than at the ends 14,16 to provide additional support to the center portion of the user's lower back, if desired.

The back support member 12 can be fabricated from a flexible material adapted to support the user's lower back when worn. An example of a flexible material can include elastic webbing such as that commonly used in the construction of back support devices. Other materials such as canvass may also be utilized. In some embodiments, the back support member 12 may be cushioned or padded, although versions where no additional cushioning or padding is provided are also contemplated. The material forming the back support member 12 can be folded upon itself one or more times and then sewn together along the ends 14,16 and sides 18,20, forming a multi-layered structure. In those embodiments where elastic webbing is used, for example, the webbing material can be folded upon itself one or more times and then sewn together along the ends 14,16 and/or sides 18,20 to form a multi-layered structure that increases the amount of back support provided to the user's back.

The back support device 10 may further include a number of elastic knee straps 24,26 extending from each end 14,16 of the back support member 12. A first (i.e. left) elastic knee strap 24 extending from the first end 14 end of the back support member 12, for example, can include a first end 28 located adjacent to an upper left portion of the back support member 12, and a second end 30 located adjacent to a lower left portion of the back support member 12. A second (i.e. right) elastic knee strap 26 extending from the second end 16 of the back support member 12, in turn, can include a first end 32 located adjacent to an upper right portion of the back support member 12, and second end 34 located adjacent to a lower right portion of the back support member 12. The first and second elastic knee straps 24,26 can each have an elongated shape having a length of about 25 to 35 inches, and more specifically, about 30 inches, although other lengths are possible.

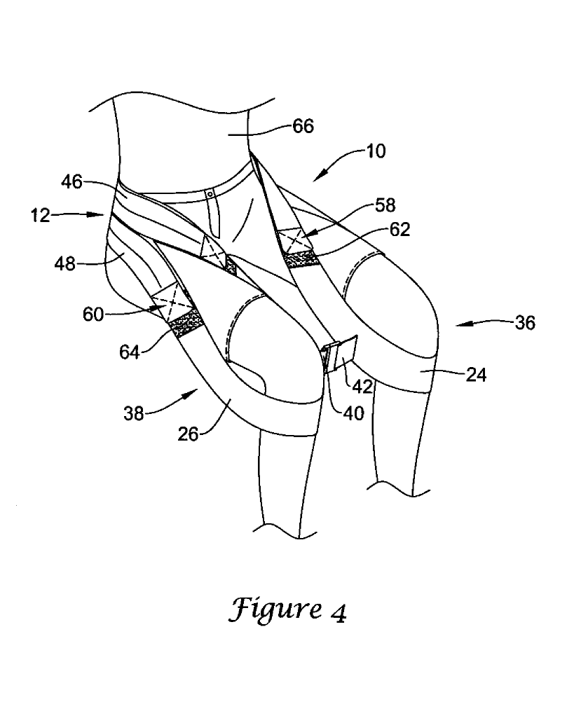

The first and second elastic knee straps 24,26 may be formed integrally as a continuous piece with the material forming the back support member 12, or can each comprise separate members that are attached to the ends 14,16 of the support member 12 by sewing, adhesion, or other suitable attachment means. For example, the first and second elastic knee straps 24,26 may be formed integrally with the elastic webbing or other such material used to construct the back support member 12. In use, and as discussed further with respect to FIG. 4, each of the elastic knee straps 24,26 can be configured to form a respective loop 36,38 that can be attached at a location approximately midway along their length to the user's knees when the user is in a seated position. If desired, the elastic knee straps 24,26 can include a padded knee brace (not shown) for even disbursement of pressure for the user's knees when the device 10 is used in a seated position.

Each of the elastic knee straps 24,26 can be connected together at a location approximately midway along their length via a fastener. In some embodiments, and as shown in FIG. 1, the fastener can include a male buckle member 40 coupled to the second elastic knee strap 26 and adapted to engage a corresponding female buckle member 42 coupled to the first elastic knee strap 24. Other fastener mechanisms such as a fabric hook and latch-type fastener (e.g. a VELCRO fastener), a clasp, and/or a drawstring or tie could also be utilized to releasably secure the loops 36,38 together. When connected, the buckle members 40,42 can be used to prevent the loops 36,38 from inadvertently slipping off of the user's knees during use. In addition, the buckle members 40,42 help to maintain the spacing of the user's legs when the device 10 is being used. In certain embodiments, one or both of the buckle members 40,42 can be made adjustable, allowing the user to adjust the separation or distance between the loops 36,38 in order to provide more or less lateral support to the user's legs.

FIG. 2 is another perspective view showing the illustrative back support member 12 of FIG. 1. As shown in FIG. 2, an interior side 44 of the back support member 12 adapted to cradle all or a portion of the user's lower back can include a number of elastic straps 46,48, which provide lumbar support to the user's lower back while also permitting the adjustment of the elastic knee straps 24,26. An upper elastic strap 46 of the back support member 12 having a first end 50 and a second end 52, for example, may extend lengthwise along the upper periphery of the back support member 12, and can be used to independently adjust the lengths of the elastic knee straps 24,26. In similar fashion, a lower elastic strap 48 of the back support member 12 having a first end 54 and a second end 56 can also be used to independently adjust the length of the elastic knee straps 24,26. The upper and lower elastic straps 46,48 can be fabricated from elastic webbing similar to that used in the construction of the elastic knee straps 24,26. In certain embodiments, for example, the upper and lower elastic straps 46,48 can include 2 or 3-inch wide elastic webbing that is sewn together.

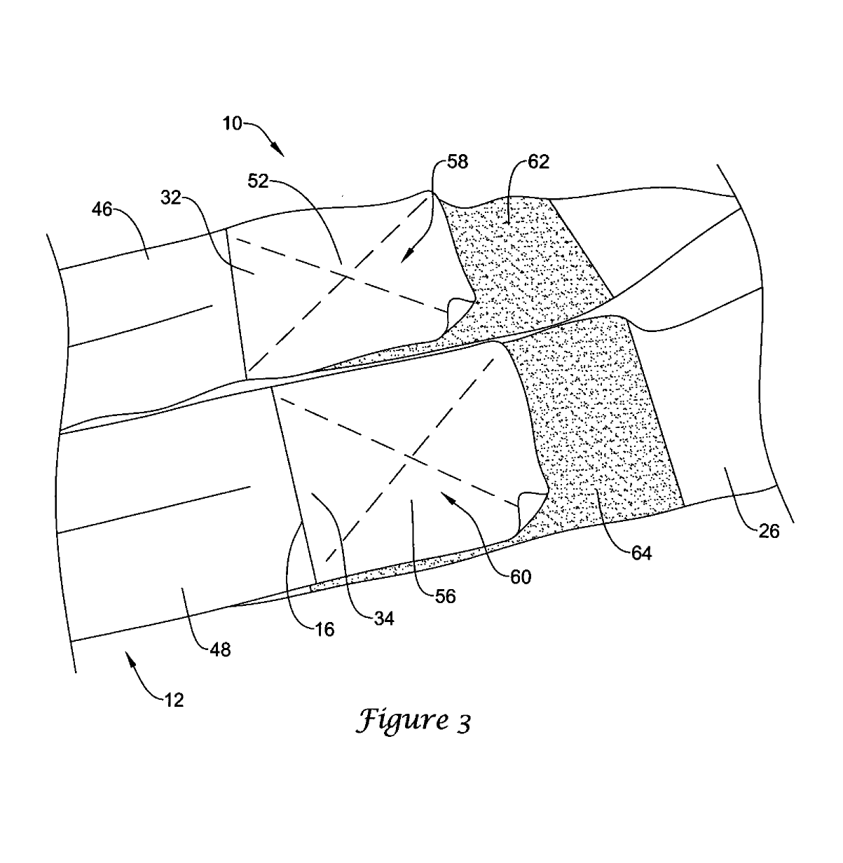

Each end 50,52 of the upper and lower elastic straps 46,48 can include an adjustment mechanism 58,60 that can be utilized to adjust the length of the elastic knee straps 24,26. As can be seen in further detail in FIG. 3, for example, the second end 52 of the upper elastic strap 46 can be made from a fabric hook (e.g. VELCRO) or the like, and can be configured to secure to a corresponding fabric receptor surface 62 formed on the end 32 of the second elastic knee strap 26. In similar fashion, the second end 56 of the lower elastic strap 48 can be made from a fabric hook or the like, and can be configured to secure to a corresponding fabric receptor surface 64 formed on the other end 34 of the second elastic knee strap 26. If desired, other types of adjustment mechanisms such as a tri-slide buckle could also be used to adjust the length of the elastic knee straps 24,26. In use, each of the adjustment mechanisms 58,60 can be used to adjust the length of the elastic knee strap 26 to the size and/or desired fit of the user. A similar set of adjustment mechanisms (not shown) can be provided on the opposite ends 50,54 of the upper and lower elastic straps 46,48 to adjust the length of the left elastic knee strap 24, as desired.

FIG. 4 is a perspective view showing a user 66 wearing the back support device 10 of FIG. 1. As shown in FIG. 4, the back support member 12 is adapted to fit against the lumbar region of the user's back with the elastic knee straps 24,26 looped over the user's knees when the user is in a seated position. When worn in this manner, the force of the user's knees pulls the back support member 12 against the lumbar region of the user's lower back, forming an elastic chair that provides back support to the user while in the seated position. In this position, the buckle members 40,42 may or may not be connected, as desired. When connected, the buckle members 40,42 maintain the knees of the user 66 together while also preventing the loops 36,38 from inadvertently detaching during use.

To vary the amount of back support provided by the device 10, or to adjust the device 10 to fit different sized users, the length of the elastic knee straps 24,26 can be adjusted using the adjustment mechanisms 58,60. To adjust the length of the right knee strap 26, for example, the user can remove one or both of the fabric hooks on the ends 52,56 of the upper and lower elastic straps 46,48 and then reposition the hooks on the fabric receptors 62,64 to either increase or decrease the length of the elastic knee strap 26, as desired. Adjustment of the left elastic knee strap 24 can be accomplished in similar fashion via the adjustment mechanisms coupled to the ends of the elastic knee strap 24.

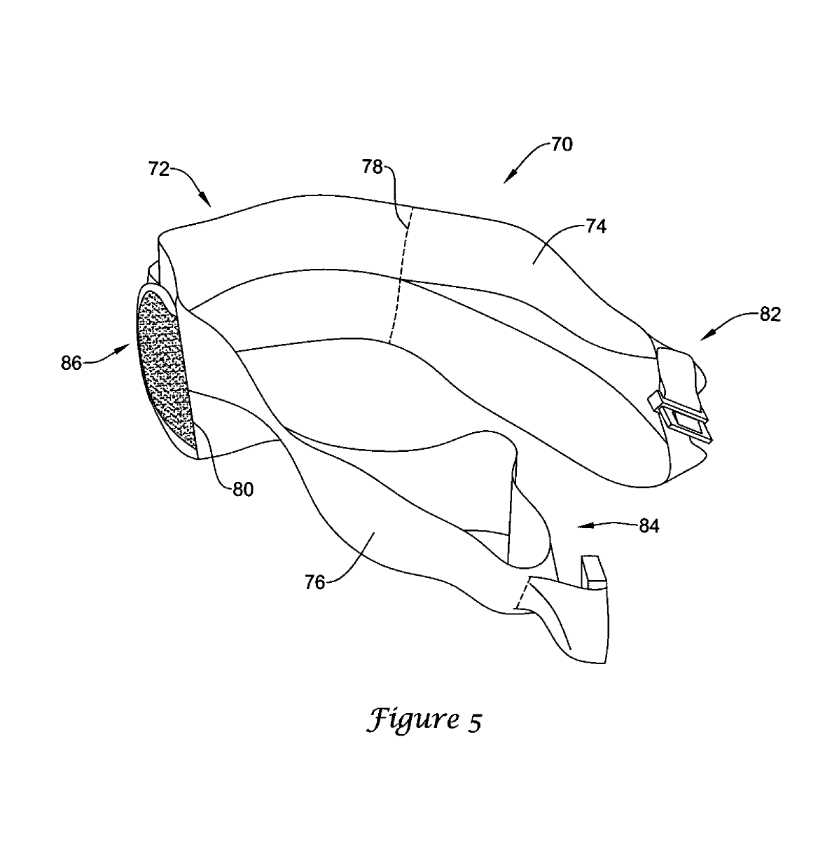

FIG. 5 is a perspective view of a back support device 70 in accordance with another illustrative embodiment having a back support adjustment mechanism. Back support device 70 is similar to the back support device 10 described above with respect to FIGS. 1-4, including a flexible, double-ended back support member 72 configured to fit adjacent to the user's lower back, and a number of elastic knee straps 74,76 extending from each end 78,80 of the back support member 72. The elastic knee straps 74,76 can each be configured to form a respective loop 82,84 that can be attached at a location approximately midway along their length to the user's knees when the user is in a sitting position, forming an elastic chair that provides back support for the lumbar region of the user's lower back.

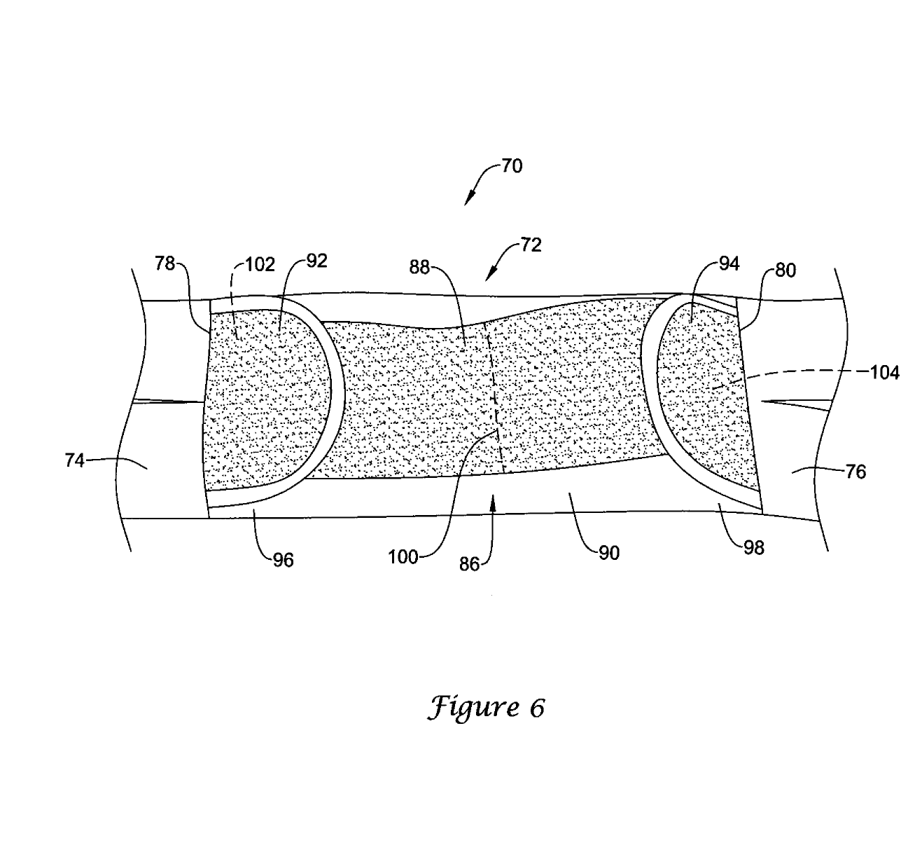

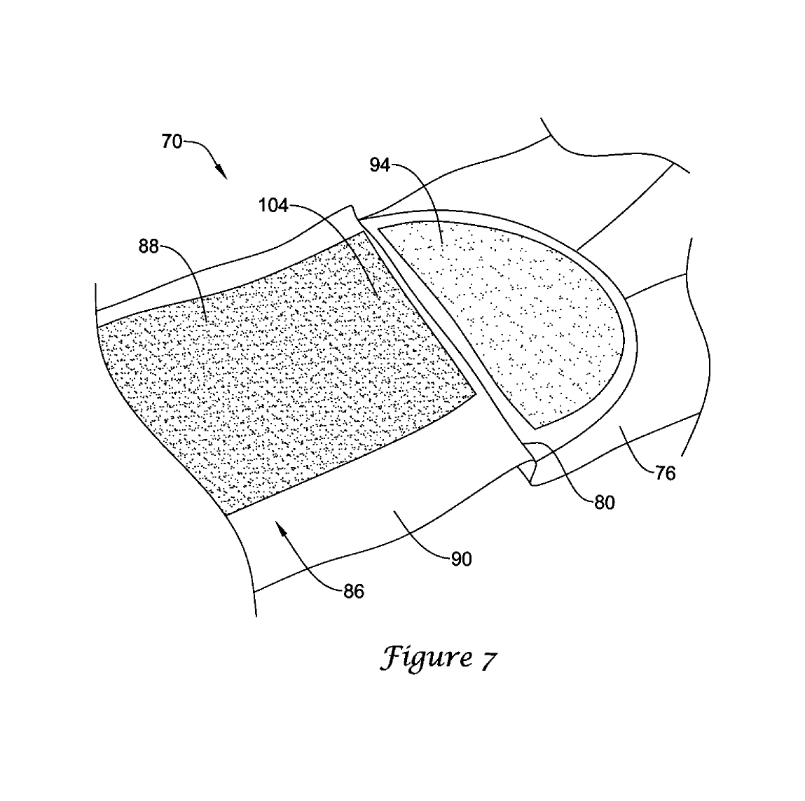

In the illustrative embodiment, the back support device 70 further includes a back support adjustment mechanism 86 that can be used to adjust the length of the back support member 72. As can be further seen in FIG. 6, for example, the adjustment mechanism 86 can include a fabric hook 88 attached to the interior side 90 of the back support member 72 and adapted to mate with several fabric receptor flaps 92,94 coupled to the ends 96,98 of the member 72. The fabric hook 88 can be secured to the center portion of the back support member 72 along a stitching line 100, and can include a pair of free ends 102,104 that are configured to mate with and receive the fabric receptor flaps 92,94. As can be seen further in FIG. 7, the underside portion of the fabric receptor flaps 92,94 can be fabricated from a material such as VELCRO that can be releasably secured to the surface of the fabric hook 88, allowing the flaps 92,94 to be releasably secured to the hook 88.

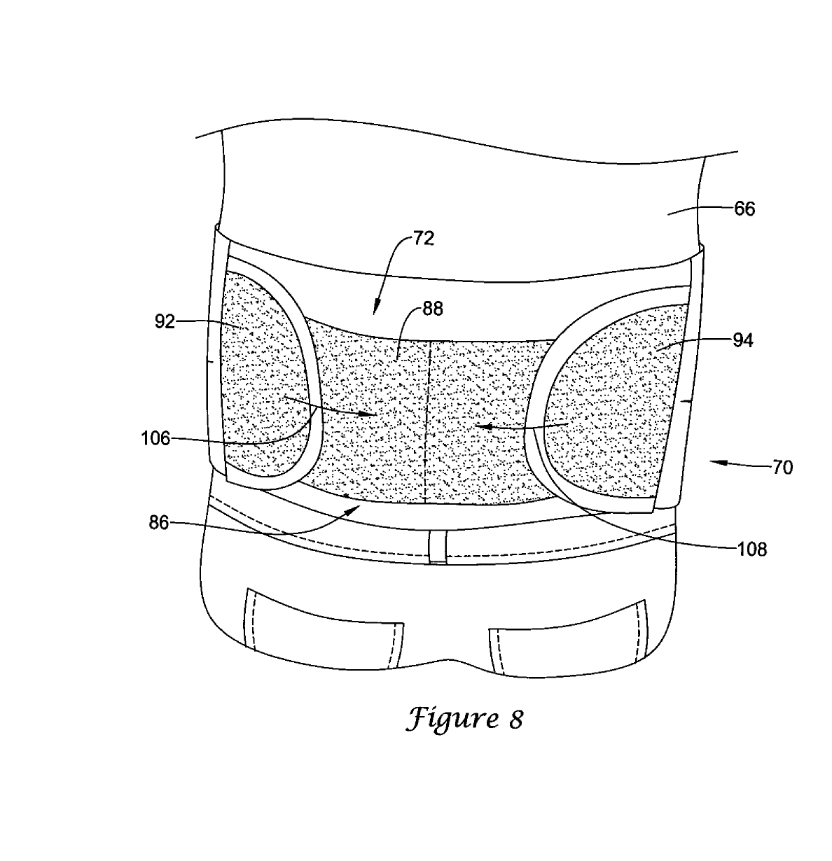

FIG. 8 is a perspective view showing the rear of the back support device 70 of FIG. 5 attached to a user's 66 body. As shown in FIG. 8, the length of the back support member 72 can be adjusted by releasing the fabric receptor flaps 92,94 from the fabric hook 88, and then either moving the flaps 92,94 either closer to or further away from the center portion of the fabric hook 88 depending on whether the user desires to decrease or increase the length of the back support member 72. If, for example, the user desires to decrease the length of the back support member 72, the user can pull the fabric receptor flaps 92,94 inwardly in the direction indicated generally by arrows 106,108, causing the length of the member 72 to shorten. By adjusting the fabric receptor flaps 92,94 relative to the fabric hook 88 in this manner, the back support member 72 can be adjusted to a number of different sizes to accommodate the size and/or wearing preferences of the user.

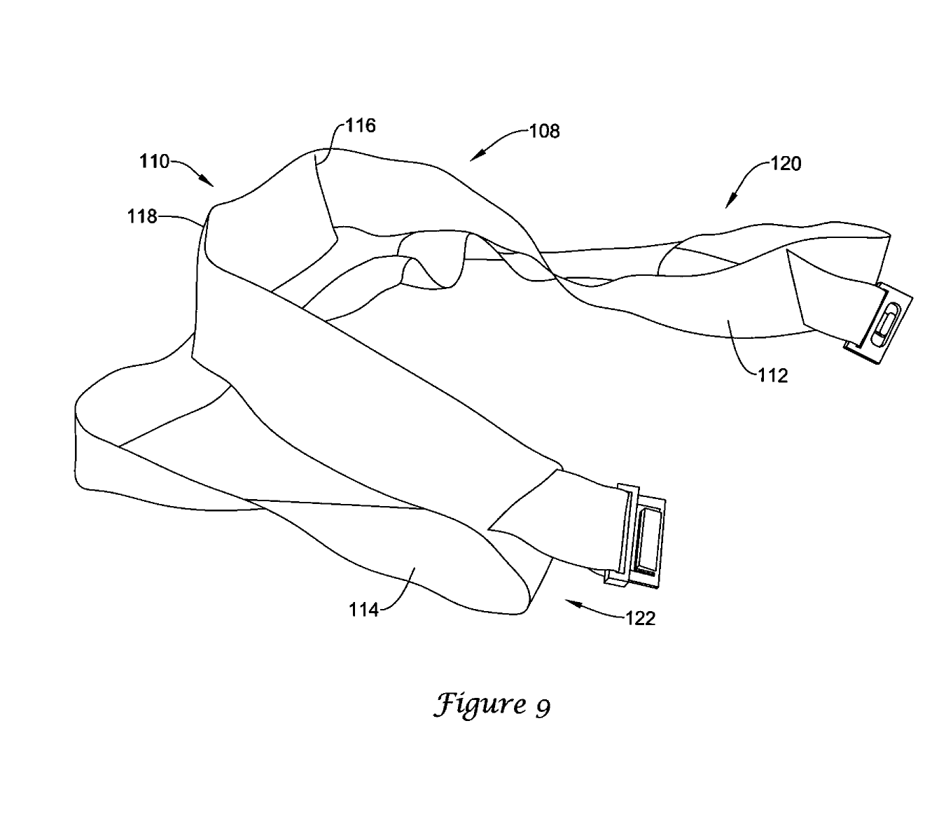

FIG. 9 is a perspective view of a back support device 108 in accordance with another illustrative embodiment having an interior pouch. The back support device 108 can be configured similar to the other back support devices 10,70 described herein, including a back support member 110 configured to fit adjacent to the user's lower back, and a number of elastic knee straps 112,114 extending from each end 116,118 of the back support member 110 and forming a respective loop 120,122 that can be attached to the user's knees.

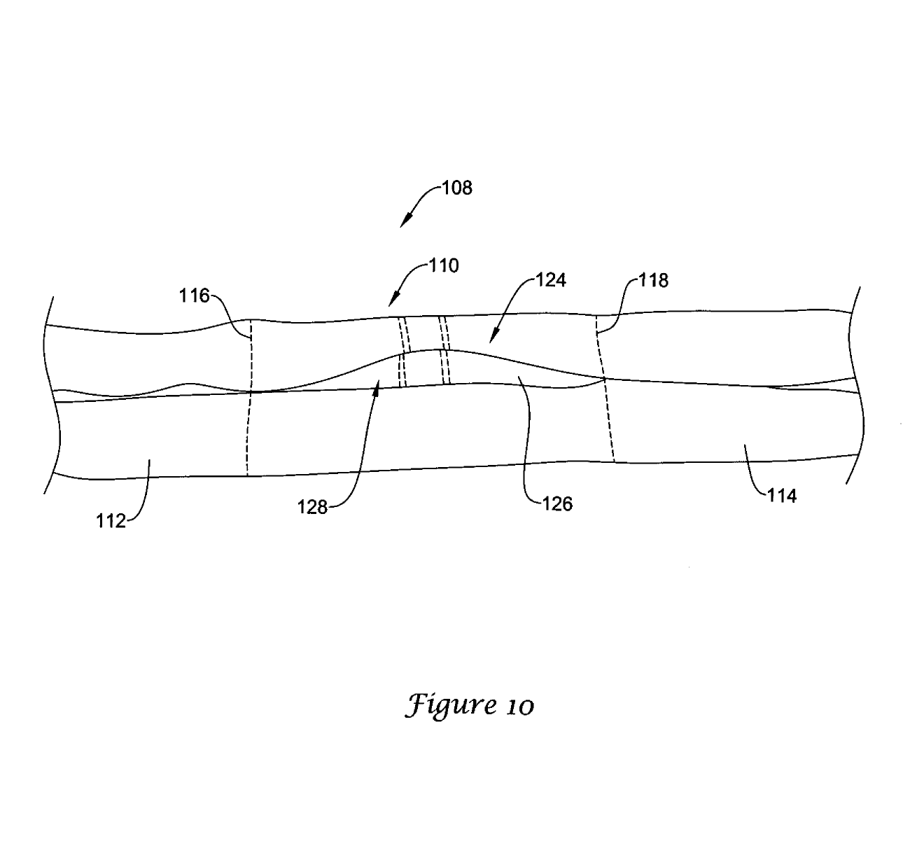

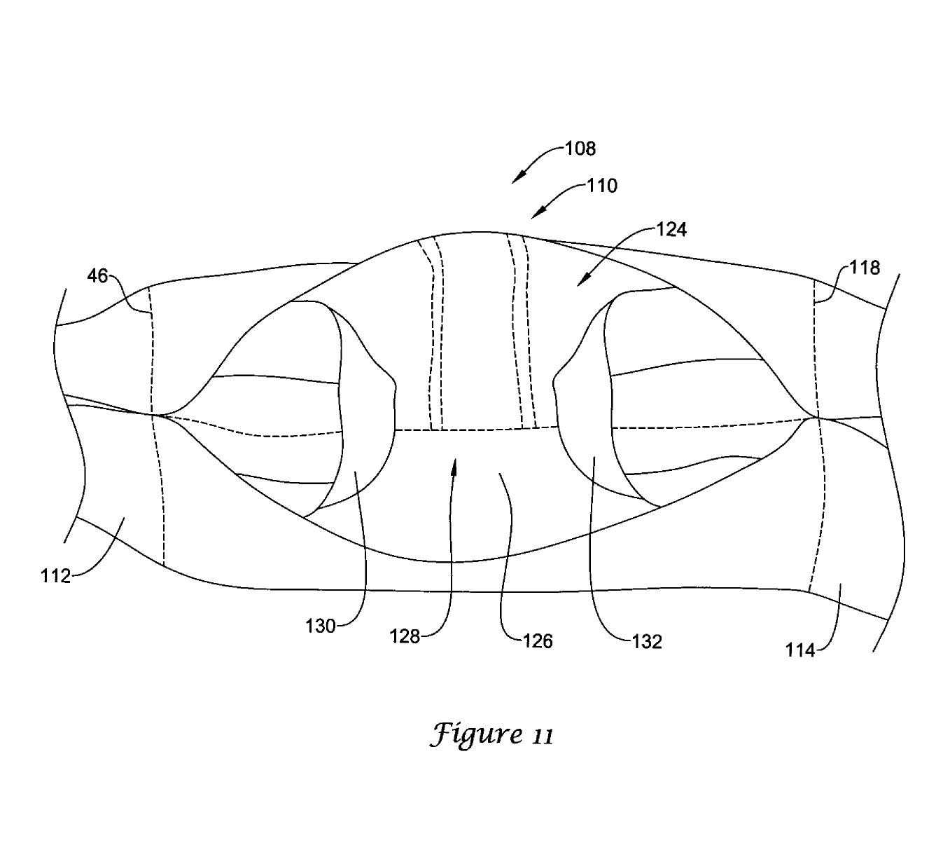

FIG. 10 is a perspective view showing an interior pouch 124 of the back support device 108 of FIG. 9. As shown in FIG. 10, the interior pouch 124 may define a cavity 126 that can be used to fold the back support device 108 upon itself when not in use, and for receiving a therapeutic device such as a thermal pack or vibration element when the device 108 is worn. In certain embodiments, the interior pouch 124 may be formed from an unsewn portion of the back support member 110, forming an opening 128 that can be used to place the therapeutic device within the cavity 126. When the back support member 110 is wrapped around the user's lower back, the opening 128 can be configured to close similar to a coin purse, holding the therapeutic device securely in place within the cavity. As shown in another view in FIG. 11 with the opening 128 fully expanded, a number of handles 130,132 within the interior pouch 124 can be provided to facilitate carrying of the back support device 108 once the device 108 is folded upon itself and collapsed.

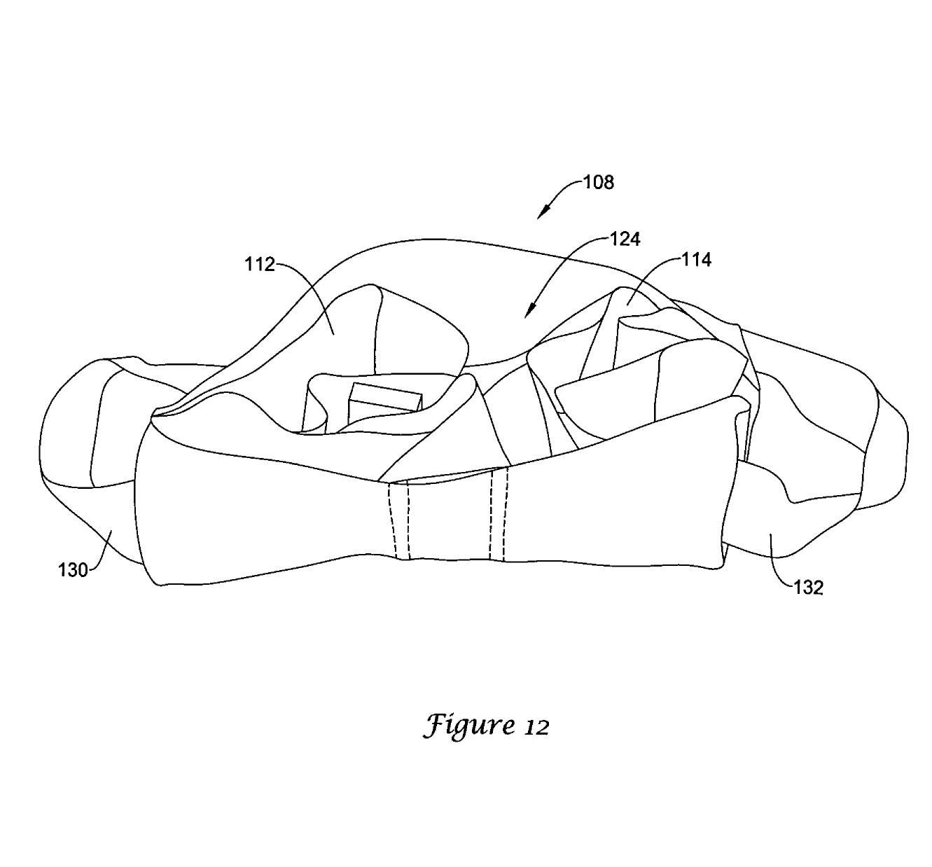

FIG. 12 is a perspective view showing the back support device 110 of FIG. 9 partially folded upon itself and collapsed within the interior pouch 124. As shown in FIG. 11, the elastic knee straps 112,114 can be folded into the interior pouch 124 for storage or transport. In this position, the handles 130,132 secured to the back support member 110 can be used to help carry the device 108.

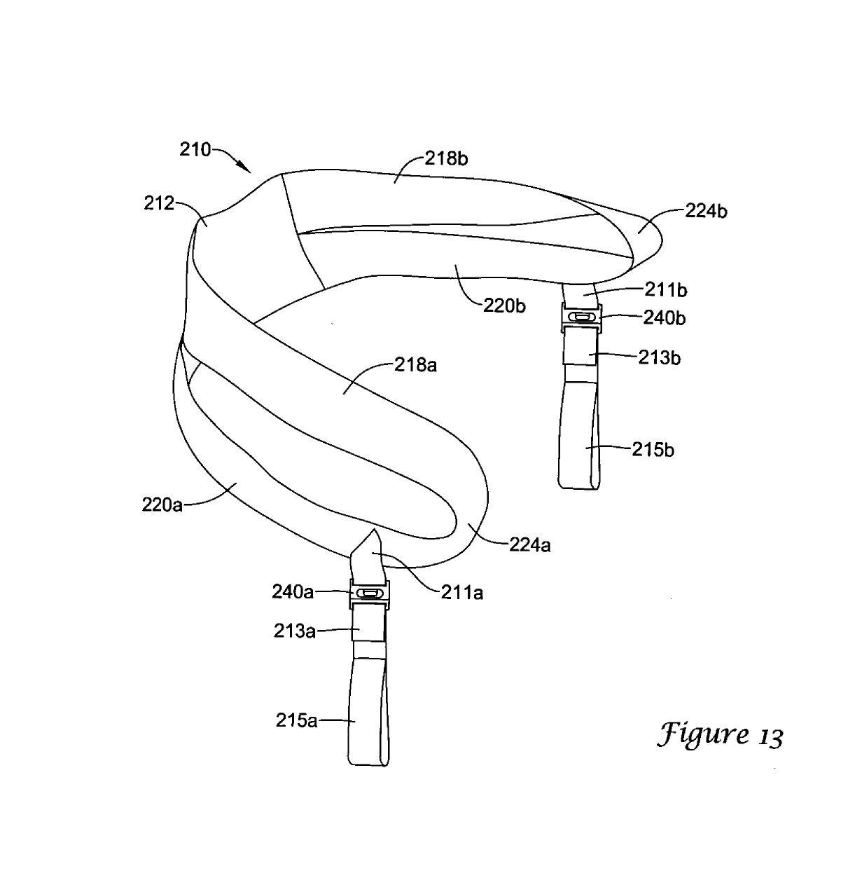

FIG. 13 is a perspective view of a back support device 210 in accordance with another illustrative embodiment, which may be similar in form and function to any of the other back support device disclosed herein. The back support device 210 may include a back support member 212. A first pair of straps 218a, 218b may extend from the back support member 212 and form a knee strap 224a. On the opposite side of the back support member 212, another pair of straps 218b, 220b may extend therefrom and form another knee strap 224b. A pair of adjustable foot straps 211a, 211b may extend from the knee straps 224a, 224b, respectively. Each of the adjustable straps 211a, 211b may include a foot loop 215a, 215b. The portion of the straps 211a, 211b extending from the foot loops 215a, 215b may loop back to a buckle member 240a, 204b. The free end 213a, 213b of the straps 211a, 211b may extend out from the buckle member 240a, 240b. In some embodiments, the rear side of the free ends 213, 213b of the straps 211a, 211b may be fitted with a fastener (e.g., a VELCRO fastener, not shown) such as a plurality of hooks (or loops) that can attach with the corresponding loops (or hooks) that may be present on the opposing side of the straps 211a, 211b. This may allow the straps 211a, 211b to be adjusted to match the size of the user. Of course any suitable fastener and/or attachment mechanism may be utilized without departing from the spirit of the invention.

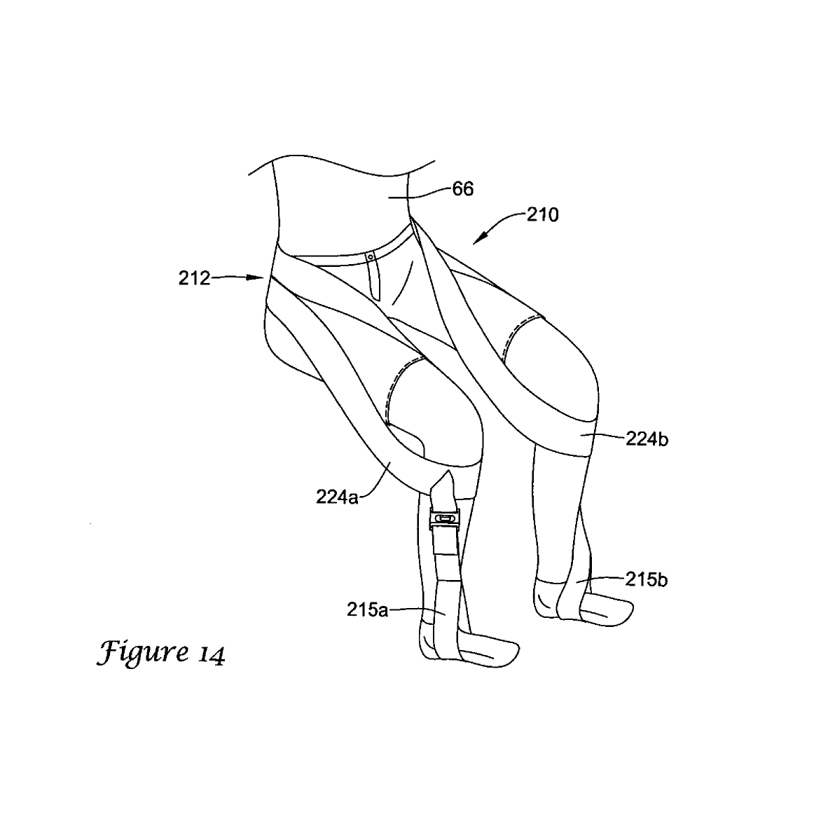

The back support device 210 may worn by a user 66 to provide the desired level of support as illustrated in FIG. 14. For example, each of the knee straps 224a, 224b may be fitted over the knees of the user 66. Likewise, each of the foot loops 215a, 215b may be fitted over the feet of the user 66.

The back support device 210 may include a number of additional structural features including any of those described above in relation to other back support devices. For example, any of straps 218a, 218b, 220a, 220b may be adjustable and may include buckles, hook and loop fasteners, other adjustment mechanisms, etc. Other straps and/or portions of the back support device 210 may similarly include features that allow the back support device 210 to be adjusted to fit essentially any user. Additionally, the back support member 212 may include a pouch (not shown), similar to interior pouch 124, that may be utilized to fold the back support device 210 in a compact form.

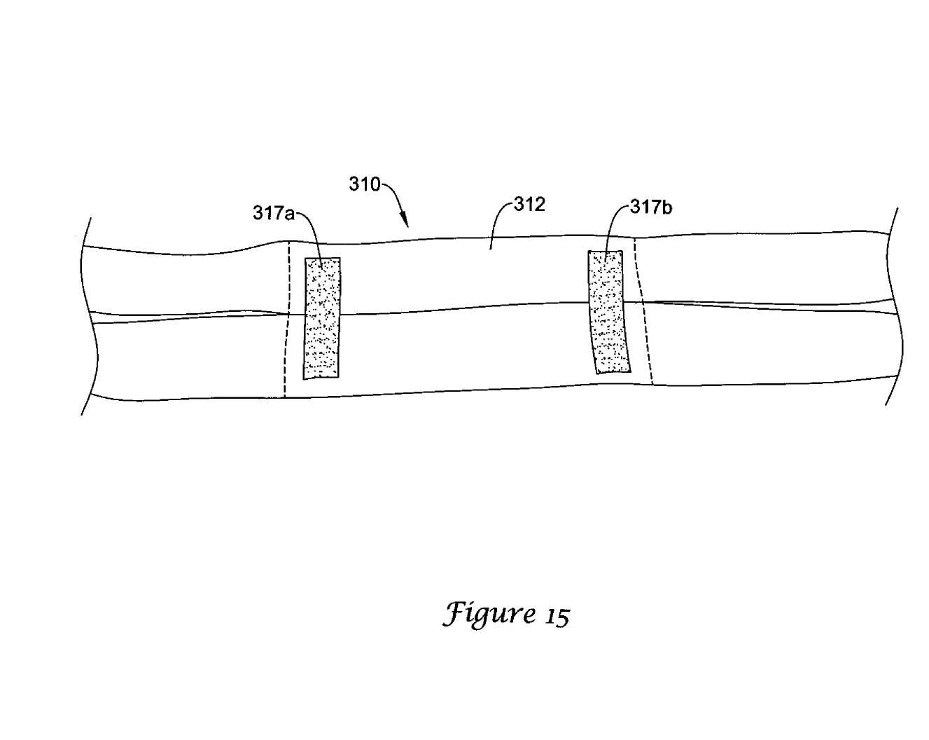

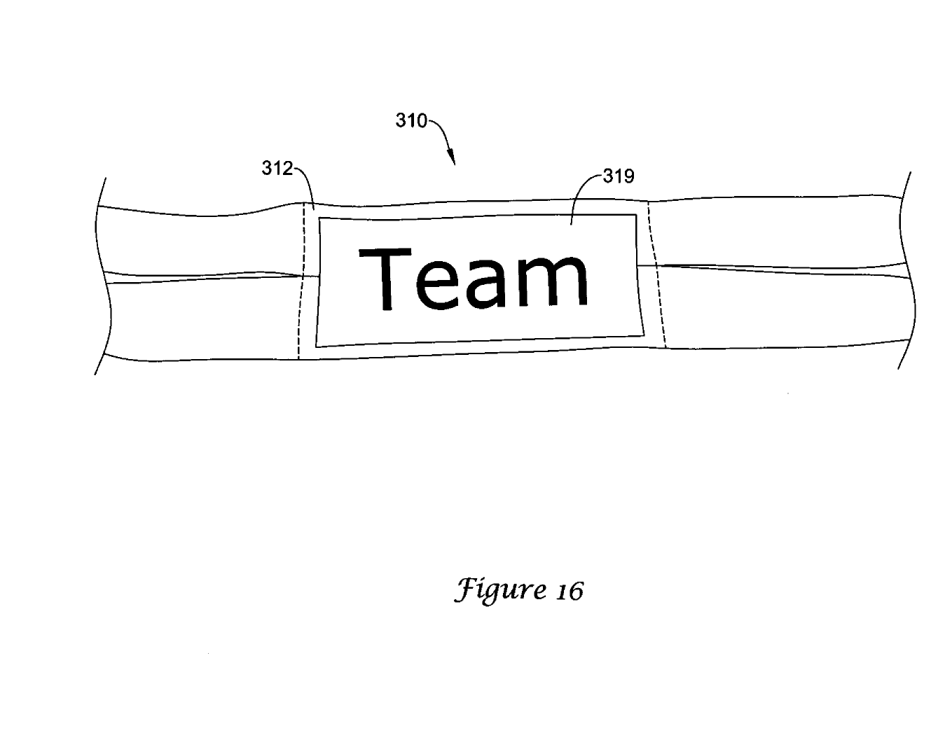

FIG. 15 illustrates another example back support device 310. The back support device 310 may include a back support member 312 with one or more fasteners 317a, 317b, that may allow an accessory member 319 (illustrated in FIG. 16) to be attached thereto. In the example illustrated in FIG. 15, the fasteners 317a, 317b may include a hook and/or loop fastener (e.g., VELCRO) that is configured to attach with the corresponding loop and/or hook fastener (not shown) disposed on the back side of accessory member 319. Other types of fasteners are contemplated for fasteners 317a, 317b including essentially any suitable type of fastener. In addition, some embodiments of the back support device 310 may include only one or more than two fasteners (e.g., three, four, five, six, seven, eight, etc.), as desired. Furthermore, any of the other back support devices disclosed herein may also include or otherwise utilize fasteners 317a, 317b and an accessory member 319.

In some embodiments, the accessory member 319 may include the logo or name of a sporting team. This may include professional sporting teams, college teams, local teams, individual players or athletes, etc. Alternatively, accessory member 319 may bear the logo, text, and/or an image that is tailored to a particular sport, leisure, or outdoor activity (e.g., hunting, fishing, tenting, canoeing, hiking, etc.). Because fasteners 317a, 317b may allow for the accessory member 319 to be removed from the back support member 312, a user may apply one accessory member 319 when attending or otherwise contemplating a particular event or team (e.g., an accessory member 319 bearing a MINNESOTA VIKINGS logo while at a MINNESOTA VIKINGS game) and then replace it with a different accessory member 319 when attending or otherwise contemplating another event (e.g., an accessory member 319 bearing a MINNESOTA TWINS logo while at a MINNESOTA TWINS game). Of course, a wide variety of accessory members 319 are contemplated that may vary as widely as the interests of the user.

While several different features are described herein with respect to specific embodiments depicted in the drawings, it is contemplated that the back support device can include any number of different combinations of features. For example, the back support device can include both an adjustment mechanism for adjusting the length of the elastic knee straps and a back support adjustment mechanism for adjusting the length of the back support member.

Having thus described the several embodiments of the present invention, those of skill in the art will readily appreciate that other embodiments may be made and used which fall within the scope of the claims attached hereto. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of the invention.

What is claimed is:

1. A back support device for supporting a user's lower back, comprising:
a double-ended back support member adapted to provide lumbar support to the user's lower back;
a plurality of elastic knee straps extending from the ends of the back support member, each of said plurality of elastic knee straps including a first end and a second end both coupled to or formed integrally with an end of the back support member and forming a loop adapted to engage a knee of the user when the user is in a seated position;
an adjustment mechanism for selectively adjusting the lengths of each of the elastic knee straps; and
a fastener for securing the elastic knee straps together at a location between said first and second ends.
